(19) United States
(12) Patent Application Publication
(10) Pub. No.: US 2026/0290618 A1
(43) Pub. Date: Sep. 24, 2026

(54) SYSTEM FOR ASSISTING TELE-PRACTICE IN TRADITIONAL CHINESE MEDICINE

(71) Applicant: Logistics and Supply Chain MultiTech R&D Centre Limited, Pok Fu Lam (HK)

(72) Inventors: Leung Chiu, Pok Fu Lam (HK); Chi Po Chong, Pok Fu Lam (HK); Chun Pong Fu, Pok Fu Lam (HK); Yuan Li Yin, Pok Fu Lam (HK); Ying Lu Xu, Pok Fu Lam (HK); Wing Leung Chow, Pok Fu Lam (HK); Ka Lun Fan, Pok Fu Lam (HK)

(21) Appl. No.: 19/086,284

(22) Filed: Mar. 21, 2025

(51) Int. Cl. G16H 80/00; G16H 20/90

(52) U.S. Cl. CPC G16H 80/00; G16H 20/90

(57) ABSTRACT

The present invention provides a system for assisting Tele-Practice in Traditional Chinese Medicine including a pulse sensing device and a pulse regeneration device. The pulse sensing device is adapted to conduct palpation by capturing pulse patterns of a person. The pulse sensing device is connected to the pulse regeneration device via a communication network. The pulse regeneration device is adapted to simulate a digital twin of the pulse patterns of the patient in real-time.

### TECHNICAL FIELD

The present invention relates to a system for assisting tele-practice in traditional Chinese medicine (TCM). More particularly, the present invention relates to a system comprising pulse sensing and pulse regeneration devices connected via a wireless network.

### BACKGROUND

The diagnostic approach within the framework of Traditional Chinese Medicine (TCM) emphasizes the significance of observation, auditory perception, inquiry, and palpation. This methodology places a considerable reliance on interpersonal interaction between practitioners and patients.

Pulse diagnosis in traditional Chinese medicine is one of the most central diagnostic techniques in the four diagnostic methods of TCM and is also the most commonly used and exploratory diagnostic means. The pulse-taking of traditional Chinese medicine is also called “pulse-feeling” and involves the use fingers of the doctors or practitioners to press the cun, guan, and chi (qi) acupoints of the cun-kou radial artery of patients so as to assess the state of an illness and distinguish the condition or disease of the patient by sensing the pulse-beating condition.

Since the COVID-19 pandemic, tele-practice has been heavily promoted. However, Traditional Chinese Medicines (TCM) tele-practice is not fully implemented because palpation is required in many TCM applications and is difficult via stand-alone video conferencing technology.

Conventionally, Chinese Medicine Practitioners (CMP) feel pressure changes by fingers in a face-to-face consultation. Without instrumental analysis, the practitioner practicing TCM, by using their expertise, can use their fingers to apply varying pressures on the patient's arteries to read pulses based on their own experiences and sensations. In turn, the CMP can then express the reading by words to form a diagnosis. However, this diagnostic step cannot be carried out remotely, and thus remote practice of TCM may be difficult without this diagnostic step.

### SUMMARY OF THE INVENTION

In accordance with an aspect of the present invention, there is provided a system for assisting tele-practice in traditional Chinese medicine (TCM).

In accordance with another aspect of the present invention, there is provided a system comprising pulse sensing and pulse regeneration devices connected via a wireless network.

In accordance with a first aspect of the present invention, there is provided a system for assisting Tele-Practice in Traditional Chinese Medicine comprising:
a pulse sensing device adapted to conduct palpation by capturing pulse patterns of a person; and
a pulse regeneration device,
wherein the pulse sensing device is connected to the pulse regeneration device via a communication network such that the pulse regeneration device is adapted to simulate a digital twin of the pulse patterns of the patient in real-time.

In a preferred embodiment, the pulse sensing device comprises an artery viewing device for imaging acupoints of a radial artery.

In a preferred embodiment, the artery viewing device comprises an infrared light source.

In a preferred embodiment, the artery viewing device comprises a controller for controlling an intensity of the infrared light source.

In a preferred embodiment, the artery viewing device comprises an arm holder for supporting an arm of the person.

In a preferred embodiment, the infrared light source is configured to be located below the arm holder.

In a preferred embodiment, the artery viewing device comprises an infrared camera.

In a preferred embodiment, the infrared camera is mounted directly above the light source.

In a preferred embodiment, the artery viewing device comprises a dynamic force control, wherein the dynamic force control is adapted to induce pressure and to collect pulse patterns.

In a preferred embodiment, the dynamic force control comprises a pulse sensor for collecting pulse patterns.

In a preferred embodiment, the dynamic force control comprises a stepper actuator for driving the pulse sensor.

In a preferred embodiment, the dynamic force control comprises a local cell controlled by a microcontroller to adjust the pressure induced by the stepper actuator.

In a preferred embodiment, the microcontroller has a feedback algorithm for limiting the pressure remains within a specified range.

In a preferred embodiment, dynamic force control comprises a linear motor for horizontal movement and an actuator connected to the pulse sensor.

In a preferred embodiment, the pulse regeneration device comprises one or more actuators for producing controlled mechanical vibrations.

In a preferred embodiment, the pulse regeneration device comprises a pipe and a skin such that the actuators are adapted to impart motion to the pipe and the skin.

In a preferred embodiment, the skin is silicone skin calibrated to replicate the tactile characteristics of human skin.

In a preferred embodiment, the pulse regeneration device comprises three actuators positioned to simulate palpation at three distinct anatomical locations of the cun, guan, and chi (qi) acupoints of the cun-kou radial artery of a person.

In a preferred embodiment, the pulse sensing device comprises a display for displaying the image captured by the artery viewing device.

In a preferred embodiment, the pulse regeneration device comprises a display for displaying the image captured by the artery viewing device in real time.

### BRIEF DESCRIPTION OF THE DRAWINGS

Embodiments of the present invention will now be described, by way of example, with reference to the accompanying drawings in which:

FIG. 1 is a schematic diagram showing a use case of a TCM tele-practice system according to an embodiment of the present invention.

FIG. 2 is an image showing an overview of a TCM tele-practice system of FIG. 1.

FIG. 3 is an image showing a radial artery viewing device of the TCM tele-practice system of FIG. 1.

FIG. 4 is an infrared image of a subject's arm showing the position of radial artery captured by the radial artery viewing device of FIG. 3.

FIG. 5 is an image of a dynamic force control of the TCM tele-practice system of FIG. 1.

FIG. 6 is a schematic diagram of a pulse sensing device of the TCM tele-practice system of FIG. 1.

FIG. 7 is a schematic diagram of the pulse sensing device of the TCM tele-practice system of FIG. 1.

FIG. 8 is an image showing pulse recording of a human subject with the pulse sensing device of the TCM tele-practice system of FIG. 1.

FIG. 9 is a schematic diagram of a pulse regeneration device of the TCM tele-practice system of FIG. 1.

FIG. 10 is a diagram showing images of a pulse regeneration device of the TCM tele-practice system of FIG. 1.

FIG. 11 is a schematic diagram showing the process flow of the measurement method of pulse generation device of the TCM tele-practice system of FIG. 1.

FIG. 12 shows an image of a standard pulse generation device for academic use.

FIG. 13 is an image of a fixture of a pulse sensor on the pulse regeneration device.

FIG. 14 shows comparison graphs of pulse waveform from golden sample and pulse waveform from the proposed pulse regeneration device with four sets of different pulses.

### DETAILED DESCRIPTION OF THE PREFERRED EMBODIMENTS

Palpation is one of the four primary diagnostic modalities in traditional Chinese medicine (TCM) and constitutes a critical component of patient assessment for Chinese medicine practitioners. This diagnostic technique involves the systematic evaluation of hemodynamic alterations, primarily through pulse examination. The arterial pulse results from cyclical expansion and recoil of the aortic wall in response to cardiac systole and diastole, generating a wave-like oscillatory phenomenon known as the pulse wave. This hemodynamic event propagates along the arterial system and is commonly assessed at the radial artery, particularly at the wrist.

Variations in cardiac output, vascular compliance, and peripheral resistance contribute to alterations in pulse characteristics, including amplitude, rhythm, and contour. In TCM, these pulse characteristics are categorized into distinct patterns, which are palpated at the cun, guan, and chi (qi) acupoints, anatomically corresponding to segments of the radial artery as recognized in Western medicine. The pulse patterns reading at the cun, guan and chi acupoints used to be carried out in person by a Chinese Medicine Practitioner or TCM doctor.

In order to assist with the practice of tele-medicine or the tele-Traditional Chinese Medicine, embodiments of the present invention provide a system configured for the deployment in a remote location, e.g., nursing homes and mobile TCM clinics. The system of the present invention is adapted to leverage 5G or WLAN wireless communication technologies to establish real-time connectivity with practitioners across geographically dispersed locations. By enabling the transmission of patient pulse data through high-speed wireless networks, the system overcomes the inherent limitations of conventional telemedicine approaches, thereby significantly enhancing the accuracy and reliability of remote diagnostic assessments.

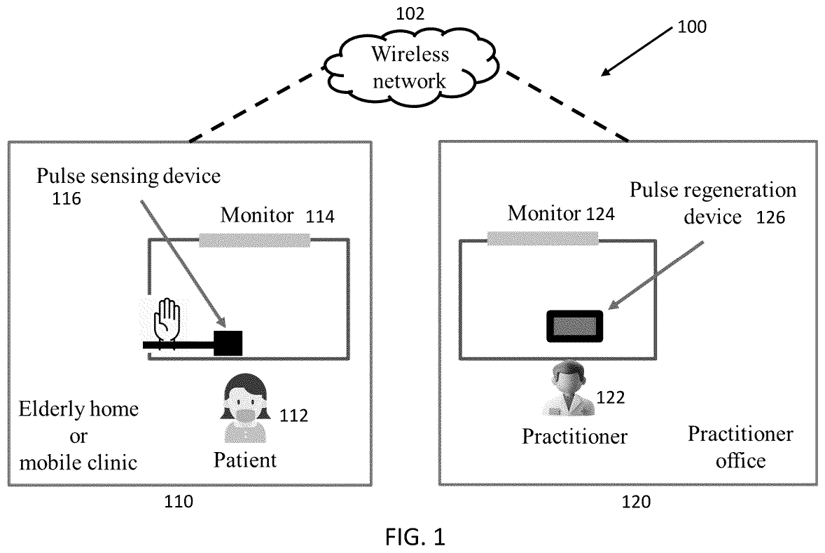

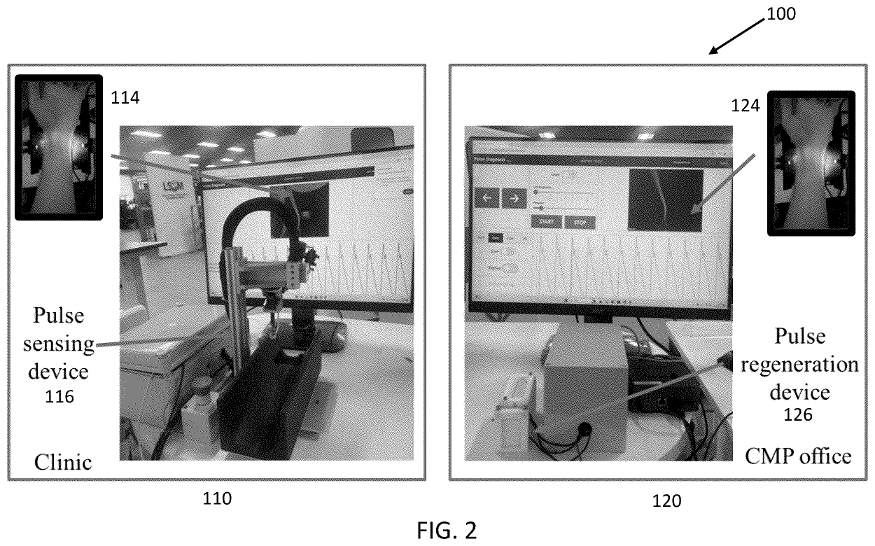

In one embodiment of the present invention, as shown in FIGS. 1 and 2, there is provided an assistive technology for TCM tele-practice in addition to the video conferencing setup. Referring to FIG. 1 and FIG. 2, the system 100 in accordance with an embodiment of the present invention comprises a remote set up 110 and a local set up 120 connected through a network 102, such as the Internet. However, the remote set up 110 and the local set up 120 may be connected through other communication means, such as wired connection. The patient's pulse data is wirelessly transmitted to the practitioner's local set up 120, enabling the practitioner to remotely feel the pulse. The system 100 of the present invention addresses the traditional limitations of tele-practice by providing crucial diagnostic information otherwise inaccessible in remote consultations. Integrating this technology significantly enhances the accuracy and reliability of remote TCM diagnostics.

The remote set up 110 has a monitor 114 and a pulse sensing device 116 for conducting palpation on a patient 112 located at the patient's site (e.g., clinic or nursing home). The local set up 120 has a monitor 124 and a pulse regeneration device 126. The local set up 120 may be situated at the TCM practitioner's office. The pulse regeneration device 126 is adapted to simulate a digital twin of the pulse patterns of the patient 112 at the remote location for the practitioner 122 to conduct diagnostics at the TCM practitioner's office. This process can be carried out in real-time or in syn.

In one embodiment of the present invention, the pulse sensing device 116 at the patient's location integrates a monitor or display screen 114 to provide real-time visualization of pulse data. The patient 112 places their arm on the pulse sensing device 116, which captures and transmits pulse patterns to the pulse regeneration device 126 via a wireless network 102.

On the practitioner's end, the pulse regeneration device 126 reconstructs and simulates the transmitted pulse patterns and presents it on a monitor or display screen 124. To ensure diagnostic accuracy, the system 100 enables the TCM practitioner 122 to remotely adjust the pulse sensor's position and pressure on the pulse sensing device 116, optimizing placement for precise pulse palpation. Since accurate pulse diagnosis depends on proper sensor positioning, this remote adjustment capability enhances the reliability of tele-TCM assessments.

Palpation is a professional procedure in TCM. Practitioners determine the palpation location on the patient's arm and the applied force based on their expertise and experience. The pulse sensing device 116 of the present invention comprises core functional components: a radial artery viewing device 202 and dynamic force control 204. These components facilitate precise localization of the palpation site and ensure controlled application of force, enhancing diagnostic accuracy and consistency.

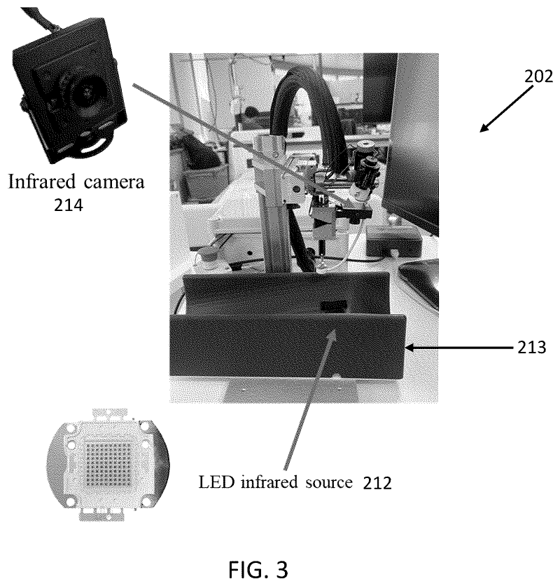

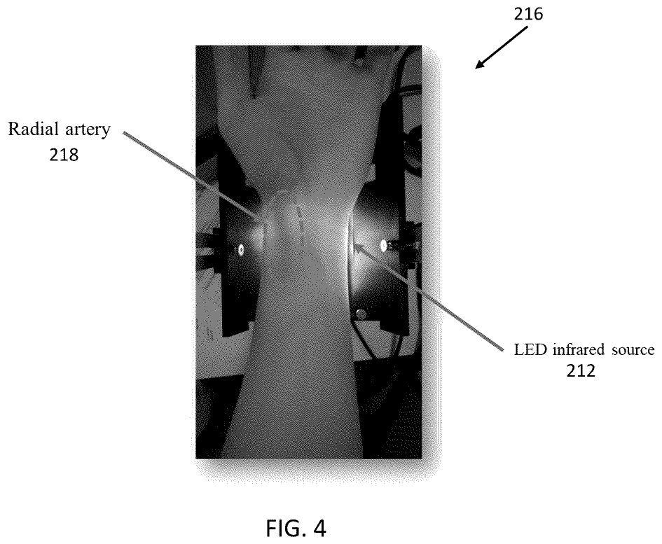

The artery viewing device 202 of the present invention employs infrared (IR) technology to enhance visualization of the patient's arm by illuminating the target region and capturing images using an infrared camera, as illustrated in FIG. 3. In contrast to the conventional blood vessel visualization device, the artery viewing device 202 of an embodiment of the present invention stabilizes the patient's arm on a dedicated support structure or arm holder 213, with an infrared light source 212 positioned beneath the arm and an infrared camera 214 mounted above infrared light source. In one embodiment, the artery viewing device has a controller for controlling the intensity and power output of the infrared light source. This configuration enables optimal image acquisition, as demonstrated in FIG. 4. The measured intensity levels for all captured images are detailed in Table 1.

TABLE 1
Different brightness levels and the corresponding
intensity and output power.

| | Intensity | Output Power |
|---|---|---|
| Level | (W/m) | (mW) |
| 1 | 1280 | 774 |
| 2 | 1400 | 847 |
| 3 | 1510 | 913 |
| 4 | 1610 | 974 |
| 5 | 1780 | 1071 |
| 6 | 1860 | 1133 |
| 7 | 1940 | 1172 |

The TCM practitioner can remotely adjust or modulate the infrared light intensity via a wireless network 100 to optimize image clarity. These images reveal the locations of blood vessels, aiding the practitioner in accurately determining the optimal pulse-taking positions. The technological innovation of the present invention significantly improves the accuracy and reliability of remote palpation and pulse diagnosis in TCM.

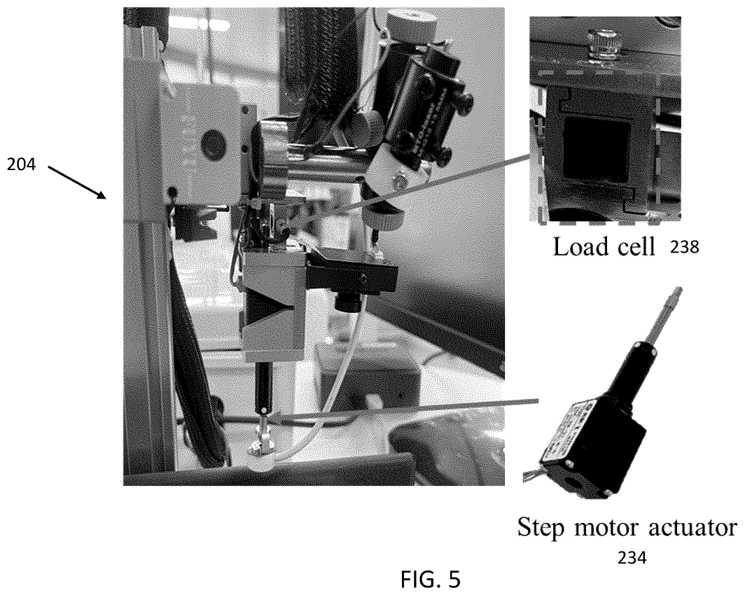

In addition to the artery viewing device 202, the pulse sensing device 116 of the present invention further comprises a dynamic force control 204. In one embodiment of the present invention, the dynamic force control 204 comprises a pulse sensor 232. The pulse sensor 232, driven by a stepper actuator 234, is adapted to induce pressure to apply on the patient's arm to collect pulse waveforms or pulse patterns, as shown in FIG. 5. The stepper actuator 234 and load cell 238 connect to a microcontroller (MCU), which uses a feedback algorithm to ensure that the applied pressure remains within a specified range. Table 2 lists all measured pressure levels. The practitioner remotely controls the pressure values.

TABLE 2
10 different pressure levels from the dynamic force control

| Force Level | Pressure Level (MPa) |
|---|---|
| 1 | 0.0008 |
| 2 | 0.0010 |
| 3 | 0.0012 |
| 4 | 0.0013 |
| 5 | 0.0016 |
| 6 | 0.0016 |
| 7 | 0.0018 |
| 8 | 0.0019 |
| 9 | 0.0020 |
| 10 | 0.0025 |

The practitioner can remotely adjust the pressure values, with the pulse waveform or pulse patterns exhibiting variability in response to different pressure settings. The practitioner then selects the optimal pressure for precise diagnostic evaluation. In another embodiment, the pulse generation device 126 comprises a sensor for sensing finger position and pressure applied on the pulse generation device and then communicates to the pulse sensing device 116. The pulse sensing device 116 then adjusts the position and pressure of the pulse sensor 232 to simulate the finger position and pressure applied on the pulse generation device 126. This process can be carried out in real time.

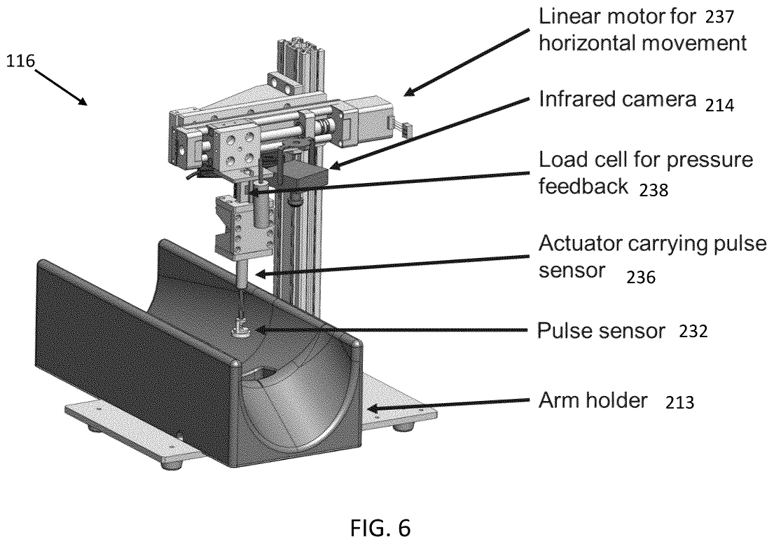

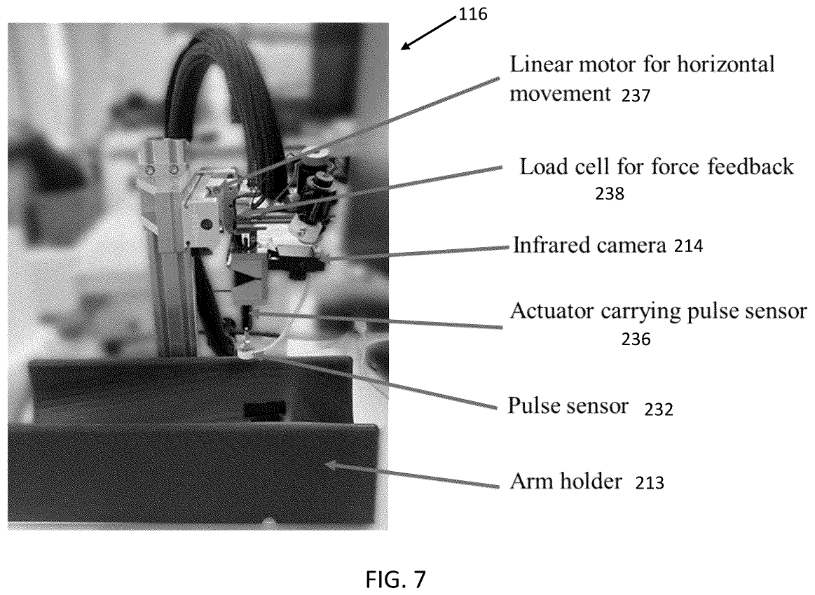

FIG. 6 and FIG. 7 show a pulse sensing device 116 of the TCM tele-practice system 100 of an embodiment of the present invention, Referring to FIG. 6 and FIG. 7, the system integration of the complete pulse sensing device 116 is presented. The pulse sensing device 116 comprises a linear motor for horizontal movement 237, a load cell for force feedback 238, an infrared camera 214, an actuator connected to the pulse sensor 236, and the pulse sensor 232, accompanied by an arm holder 213.

In one embodiment, the linear motor for horizontal displacement 237 is adapted to control precise lateral positioning of the pulse sensor 232 to align with the optimal palpation site on the patient's arm. The load cell for force feedback 238 is adapted to detect the pressure applied and provide real-time feedback to the MCU, such that the pulse sensing device 116 controls and provides consistent force application during palpation. The Infrared (IR) Camera 214 is adapted to capture high-resolution images of the patient's forearm, utilizing an infrared light source 212 to enhance the visibility of underlying blood vessels, thereby facilitating accurate sensor placement. The actuator connected to the pulse sensor 236 is a stepper motor-driven actuator 234 that dynamically adjusts the vertical displacement of the pulse sensor, modulating the applied pressure in accordance with pre-defined diagnostic parameters. The pulse sensor 232 is adapted to sense the pulse patterns or pulse waveforms of a patient. The arm holder 213 is adapted to maintain stability and minimize motion artifacts during palpation.

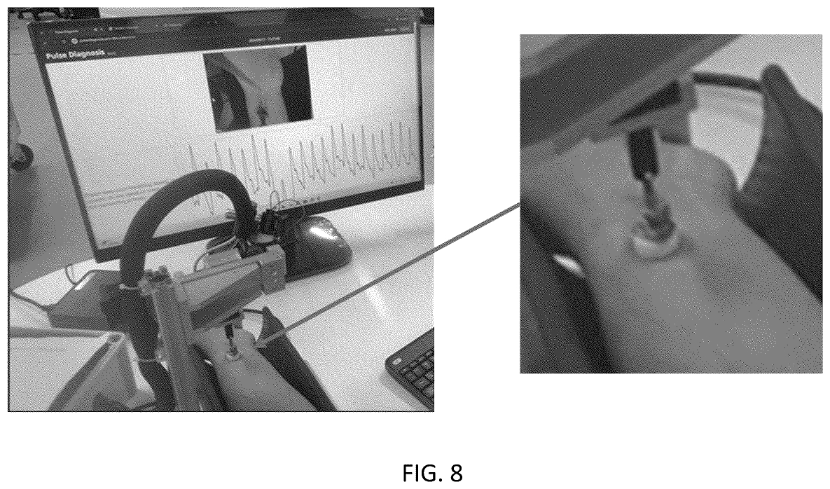

As shown in the image of FIG. 8, pulse sensing device 116 of the present invention is employed in a subject test, successfully acquiring a high-fidelity pulse waveform. The integration of precise positioning, real-time force modulation, and enhanced vascular imaging significantly improves the accuracy of pulse diagnosis in traditional Chinese medicine (TCM), facilitating both in-person and remote diagnostic applications.

The pulse regeneration device 126 of the present invention is designed to generate a digital twin of pulse patterns recorded from the pulse sensing device 116. The pulse regeneration device 126 comprises one or more actuators 252 to produce controlled mechanical vibrations, accurately replicating the pulsations of the human pulse. In one embodiment, the actuator 252 is a servo-motor-based actuator.

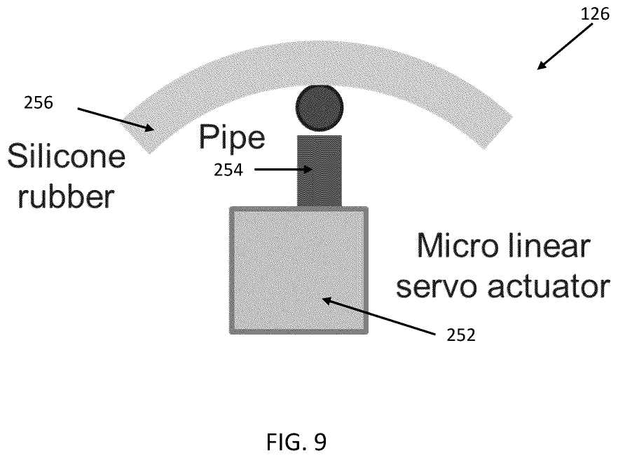

The structure of pulse regeneration device 126 according to an embodiment of the present invention is shown in FIG. 9. The pulse regeneration device 126 comprises one or more actuators 252 adapted to impart motion to a plastic tube or pipe 254 and a silicone skin layer 256. The silicone skin 256 has a mixing ratio of raw materials designed to simulate the appearance, texture, and mechanical properties of human skin. The plastic tube or pipe 254 is adapted to generate pulses similar to those from a radial artery. The silicone skin layer 256 is calibrated to replicate the tactile characteristics of human skin with a high degree of accuracy.

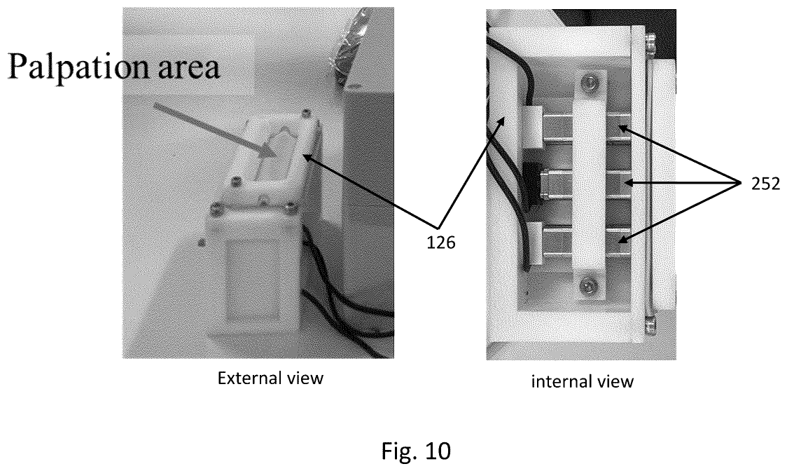

To evaluate the performance of the pulse regeneration device 126, an engineering embodiment of the present invention is constructed, as shown in FIG. 10. This embodiment comprises three actuators 252, strategically positioned to simulate palpation at three distinct anatomical locations of the cun, guan, and chi (qi) acupoints of the cun-kou radial artery of a person.

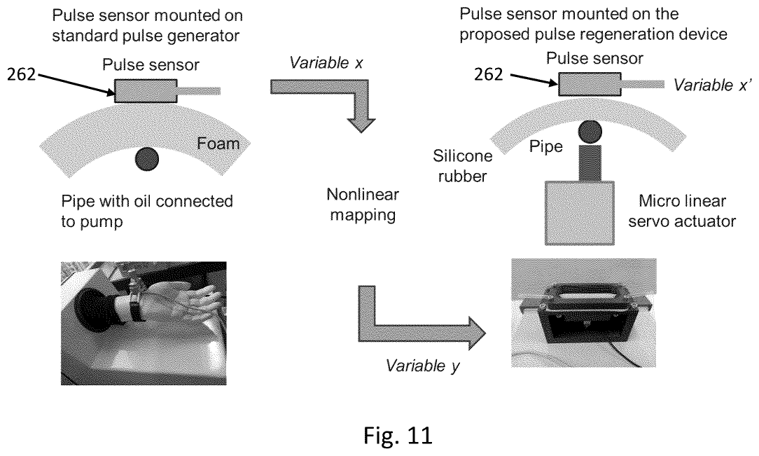

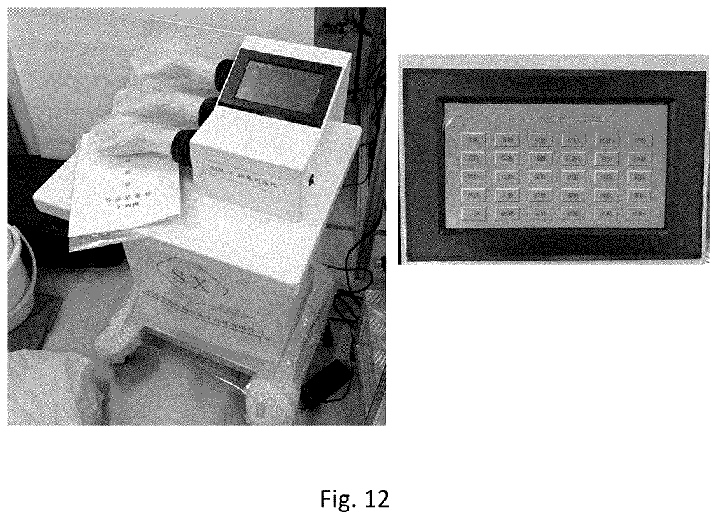

The similarity between original and regenerated pulses is compared using an engineering methodology. A systematic measurement method, as shown in FIG. 11, employs two pulse sensors 262 of model of HK-2000 to collect data. One pulse sensor 262 is affixed to a standard pulse generation device, used academically as a “golden sample” for constant pulse input as shown in FIG. 12. The input x is digitized pulse data of the “golden sample” and x represents dynamic pressure levels. This can be modeled as a time series with values ranging from 0 to 255 at a frequency of 200 Hz. The output y is a digital signal of position control for servo motor-based actuator (model: LAS10) and range of 0-2000, where 0 and 2000 represent 0 and 10.0 mm displacements, respectively. The nonlinear relationship between the input and output signals is mathematically characterized by a Taylor series expansion, as expressed in Equation (1) below.

$$y = {a}_{0} + {a}_{1} x + {a}_{2} {x}^{2} + {a}_{3} {x}^{3} + ⋯ \qquad ( 1 )$$

wherein a0, a1, a2 . . . are real numbers.

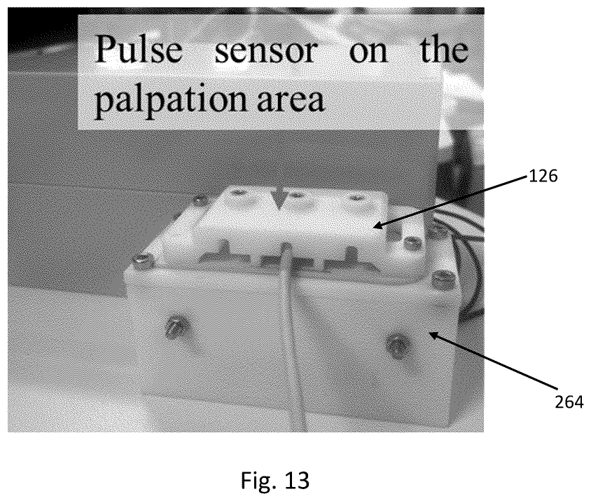

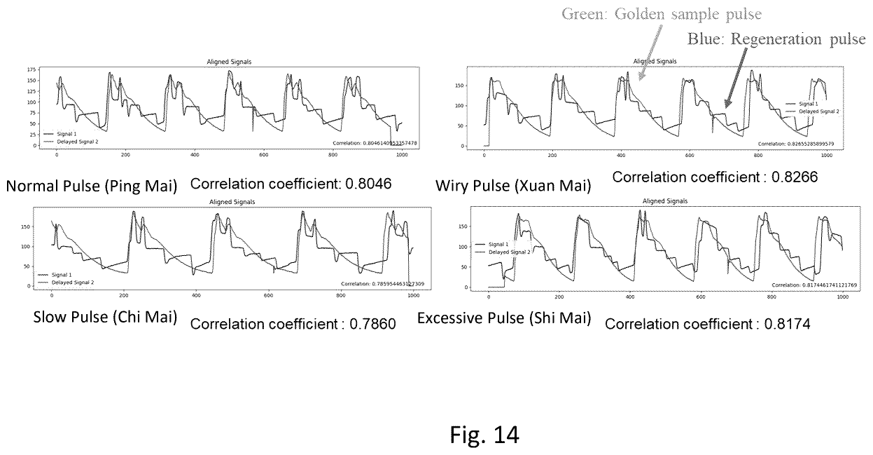

This pulse measurement model is adapted to ensure consistent pulse measurements, which cannot be reliably obtained from real subjects. Another pulse sensor 262 with digitized pulse data of x′ is also required for comparative analysis. This pulse sensor 262 is affixed to the pulse regeneration device 126 by a fixture 264 as shown in FIG. 13. Subsequently, two pulse waveforms, denoted as x and x′, shall be collected, and their correlation coefficient (r) shall be calculated in accordance with Equation (2).

$$r = \frac{{\sum }_{i = 1}^{n} ( {x}_{i} - \overline{x} ) ( {{x}^{′}}_{i} - \overline{{x}^{′}} )}{\sqrt{{\sum }_{i = 1}^{n} {( {x}_{i} - \overline{x} )}^{2}} \sqrt{{\sum }_{i = 1}^{n} {( {{x}^{′}}_{i} - \overline{{x}^{′}} )}^{2}}} \qquad ( 2 )$$

wherein n is number of data. In this measurement, n=1000 (in 5.0 sec) is selected 4-6 clear cycle of pulse waveforms is observed. In one embodiment, Equation (1) is considered up to second order terms, variables a₀, a₁ and a₂ are optimized to maximize r. Eventually, a₀=600, a₁=0.3 and a₂=0.08 are chosen. Four common and available pulse types are selected, with correlation coefficients ranging from 0.7860 to 0.8266 as shown in FIG. 14.

The present invention provides an innovative TCM tele-practice system 100, integrating pulse sensing and regeneration devices connected wirelessly. The system 100 enables remote pulse diagnosis, enhancing telemedicine diagnostics by transmitting and regenerating pulse data with a high correlation coefficient between real and regenerated pulses. This technology promises significant improvements in diagnostic accuracy and patient care in TCM tele-practice. Future work will focus on refining system components and validating their clinical efficacy.

The present invention discloses a novel and inventive TCM tele-practice system 100 which integrates pulse sensing and regeneration devices interconnected via wireless communication. The system 100 facilitates remote pulse diagnosis, thereby advancing telemedicine diagnostics through the transmission and regeneration of pulse data with a high correlation coefficient between real and regenerated pulses. This technological advancement is expected to significantly enhance diagnostic precision and patient care within the scope of TCM tele-practice. Further improvement can be achieved by refining system components and validating their clinical efficacy.

It will be understood by those skilled in the field that various modifications and alternatives can be made to the embodiments described above, without deviating from the overall scope of the present disclosure. These variations may include, but are not limited to, changes in specific implementations, configurations, or methodologies, provided that they remain within the general principles and objectives of the invention. Consequently, the embodiments provided should be regarded as exemplary rather than limiting, and any alterations that do not alter the core functionality and purpose of the invention are considered within the scope of the present disclosure.

1. A system for assisting Tele-Practice in Traditional Chinese Medicine comprising:
a pulse sensing device adapted to conduct palpation by capturing pulse patterns of a person; and
a pulse regeneration device,
wherein the pulse sensing device is connected to the pulse regeneration device via a communication network such that the pulse regeneration device is adapted to simulate a digital twin of the pulse patterns of the patient in real-time.

2. A system of claim 1, wherein the pulse regeneration device comprises one or more actuators for producing controlled mechanical vibrations.

3. A system of claim 2, wherein the pulse regeneration device comprises a pipe and a skin such that the actuators are adapted to impart motion to the pipe and the skin.

4. A system of claim 3, wherein the skin is silicone skin calibrated to replicate the tactile characteristics of human skin.

5. A system of claim 4, wherein the pulse regeneration device comprises three actuators positioned to simulate palpation at three distinct anatomical locations of the cun, guan, and chi (qi) acupoints of the cun-kou radial artery of a person.

6. A system of claim 5, wherein the pulse sensing device comprises an artery viewing device for imaging acupoints of a radial artery.

7. A system of claim 6, wherein the artery viewing device comprises a dynamic force control, wherein the dynamic force control is adapted to induce pressure and to collect pulse patterns.

8. A system of claim 7, wherein the artery viewing device comprises an infrared light source.

9. A system of claim 8, wherein the artery viewing device comprises an arm holder for supporting an arm of the person.

10. A system of claim 9, wherein the infrared light source is configured to be located below the arm holder.

11. A system of claim 10, wherein the artery viewing device comprises an infrared camera.

12. A system of claim 11, wherein the infrared camera is mounted directly above the light source.

13. A system of claim 12, wherein the dynamic force control comprises a pulse sensor for collecting pulse patterns.

14. A system of claim 13, wherein the dynamic force control comprises a stepper actuator for driving the pulse sensor.

15. A system of claim 14, wherein the dynamic force control comprises a local cell controlled by a microcontroller to adjust the pressure induced by the stepper actuator.

16. A system of claim 15, wherein the microcontroller has a feedback algorithm for limiting the pressure remains within a specified range.

17. A system of claim 16, wherein dynamic force control comprises a linear motor for horizontal movement and an actuator connected to the pulse sensor.

18. A system of claim 17, wherein the artery viewing device comprises a controller for controlling an intensity of the infrared light source.

19. A system of claim 18, wherein the pulse sensing device comprises a display for displaying the image captured by the artery viewing device.

20. A system of claim 19, wherein the pulse regeneration device comprises a display for displaying the image captured by the artery viewing device in real time.
